(19) United States
(12) Patent Application Publication
(10) Pub. No.: US 2022/0127202 A1
(43) Pub. Date: Apr. 28, 2022

(54) PROCESS FOR OBTAINING A LIQUID FOLIAR FERTILIZER AND LIQUID FOLIAR FERTILIZER COMPOSITION

(71) Applicant: Hélio Akinaga HATTORI, São Paulo (BR)

(72) Inventor: Hélio Akinaga HATTORI, São Paulo (BR)

(21) Appl. No.: 17/428,839

(22) PCT Filed: Aug. 13, 2019
(86) PCT No.: PCT/BR2019/050338

§ 371 Date: Aug. 5, 2021

(30) Foreign Application Priority Data
Feb. 5, 2019 (BR) 10 2019 002365 1

(51) Int. Cl. C05F 11/00; C05G 5/23; C05C 11/00; C05G 1/00

(52) U.S. Cl. CPC C05F 11/00; C05G 1/00; C05C 11/00; C05G 5/23

(57) ABSTRACT

The present invention refers to a process for providing a liquid fertilizer for foliar application with a final formulation comprising ricinoleic acid saponified with sulfuric acid, triethanolamine and water. The invention provides a means of accelerating vegetative growth through a proposed composition that has a group of fatty acids and amino acids essential to plant development. The invention also provides foliar metabolic regulatory means without the presence of toxic components, being suitable for foliar application in all kinds of crops.

### TECHNICAL FIELD

The present invention refers to the area of Chemistry applied to agribusiness, more particularly to a product obtained from organic compounds that has application in the agricultural area as compost or fertilizer.

### STATE OF THE ART

Castor bean cake is obtained from the seeds of the generic name castor bean oilseed (Ricinus digitatus Nor., Ricinus gibsoni Cf., Ricinus leucocarpus Bert., Ricins hidrdus Bess), and represents a by-product of the castor bean production chain, the main product being castor oil also called mammon oil. The term “mammon” originates from the term “quimbundo mumono”, and thanks to the similarity of its leaves to the papaya tree, it was also influenced by the term “papaya”.

Castor bean cake has been used around the world as an organic fertilizer to recover depleted land.

Castor oil is rich in fatty carboxylic acids, particularly in ricinoleic acid (80% to 90°/), which provides good viscosity and low temperature solubility in alcohol.

Ricinoleic acid or 12-hydroxy-9-cis-octadecenoic acid is industrially extracted through saponification or fractional distillation of hydrolyzed castor oil.

The saponification reaction receives this name, due to being widely used in soap making. The reaction is characterized by the basic hydrolysis of lipids (or triglycerides, or vegetable oils, or fats), by adding a strong base and heating. Triglyceride molecules break down to form a glycerin molecule and three fatty carboxylic acid molecules in the form of a salt. If the base used is sodium, such as sodium hydroxide, the salt will be a sodium salt.

Fatty carboxylic acid salts have the property of dissolving in either polar media such as water or non-polar media. This is why the “soap” obtained at the end has the property of a surfactant.

The basic saponification reaction equation is shown below:

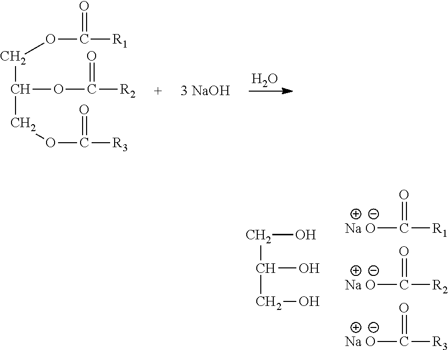

The use of ricinoleic acid in fertilizers is already known.

The document CN 105948980 refers to special fertilizer for grapes containing castor bean in its cake composition.

GB 1178849 relates to a granular fertilizer whose granulating agent can be a monoricinoleate or ricinoleic acid.

CN 104529659 describes a fertilizer that uses ricinoleic acid.

CN 106431644 teaches a selenium-rich fertilizer containing ricinoleic acid.

But none of the prior art documents teach a composition for liquid foliar fertilizer that is easy to apply and has good absorption properties by the plant.

### Invention Objects

The main object of the invention is to provide a product derived from ricinoleic acid with the addition of triethanolamine (C₆H₁₅NO₃) capable of being used as a liquid foliar fertilizer to accelerate vegetative growth.

Ricinolec acid has inherent antibacterial and antifungal properties.

### GENERAL DESCRIPTION OF THE INVENTION

The present invention refers to a process for obtaining a liquid fertilizer for foliar application with a final formulation comprising:
ricinoleic acid saponified with sulfuric acid;
triethanolamine (C₆H₁₅NO₃);
Water.

The invention provides a means of accelerating vegetative growth through the proposed composition that has a group of fatty acids and amino acids essential to plant development.

The invention also provides foliar metabolic regulatory means without the presence of toxic components, being useful for foliar application in all kinds of plants.

### DESCRIPTION OF THE FIGURES

The invention will be better understood through the description of a preferred, non-limiting exemplary embodiment, and the figures that refer to it, in which:

FIG. 1 illustrates a way of foliar application of the fertilizer of the invention, by spraying the product on plants.

FIG. 2 illustrates another way of applying the fertilizer product of the invention, by spraying on plants.

### DETAILED DESCRIPTION OF THE INVENTION

The present invention refers to a process for obtaining a liquid fertilizer composition, for foliar application, comprising the following steps:
(a) in a first container dilute a strong base in water until reaching a mass concentration of about 5% to about 25% at a temperature between about 30° C. and about 50° C. and stir until complete dissolution;
(b) add castor oil in the ratio of about 70% to about 90% of the mass of item (a) above and an alcohol in the ratio of about 3% to about 12% of the mass of item (a) above, stirring strongly, leaving to rest and then stirring again;
(c) in a second container dilute sulfuric acid in water up to about 40% to about 65% by weight to a temperature of 0° C.;
(d) without stirring add to the mixture of the second container the mixture of the first container, followed by stirring;
(e) stop the stirring and leave to rest; when phase separation occurs drain the lower phase which will be discarded;
(f) to the product obtained in step (e) add water in an amount of about 30% to about 55% of the mass of item (a) above, stir and leave to rest; phase separation will occur, drain the lower phase (waste) which will be discarded;
(g) to the product obtained in the previous step add water in an amount of about 30% to about 55% of the mass of item (a) above, stir, and leave to rest; after phase separation, discard the lower phase;
(h) add triethanolamine in a container, then water and later the product obtained in the previous step (g), in the following ratio by mass:
   2 parts of ricinoleic acid saponified with sulfuric acid;
   1 part of triethanolamine (C₆H₁₅NO₃)
   2 to 10 parts of water.

The strong base used in step (a) can be an alkali metal hydroxide such as sodium hydroxide.

Alcohol can be an alcohol containing C2 to C6, such as ethanol.

Preferably, the process of obtaining a liquid fertilizer composition for foliar application of the invention comprises the following steps:
(a) in a container dilute 100% sodium hydroxide in flakes in water to reach a concentration of 13.04% by mass at a temperature between about 35° C. and about 45° C. and stir until dissolved;
(b) add extra pale castor oil in the ratio of 82.61% of the mass of item (a) above and ethanol in the ratio of 6.52% of the mass of item (a) above, under stronger stirring for 15 min, leave to rest for 10 min and then stir again for 25 min;
(c) in another container dilute 98% sulfuric acid in water to reach a mass concentration of 52.27% at a temperature of about 0° C.;
(d) add to the mixture obtained in the previous step (c) to the mixture obtained in step (b), without stirring, and then stir for 5 min;
(e) turn off the stirring and leave to rest for 10 to 20 min, when phase separation will occur; then drain and discard the lower phase;
(f) to the product obtained in step (e) add water in an amount of 43.48% of the mass of item (a) above, stir for 15 min and leave to rest for 10 to 20 min (or the time required for phase separation); then drain and discard the lower phase;
(g) to the product obtained in the previous step, water is added in an amount of 43.48% of the mass of item (a) above; stir for 15 min, then leave to rest for 30 to 90 min, and then drain and discard the lower phase;
(h) pour triethanolamine in a container, then water and later the product obtained in the previous step (g), in the following ratios by mass:
   2 parts of ricinoleic acid saponified with sulfuric acid;
   1 part of triethanolamine (C₆H₁₅NO₃);
   2 to 10 parts of water.

Said ricinoleic acid is saponified with 98% sulfuric acid, thus receiving the name of Ricinoleic Sulfuric Acid. This process differs from the common process that uses hydrochloric acid.

The method according to the invention ultimately results in a fertilizer product for foliar application, the composition comprising the following compounds:
ricinoleic acid saponified with sulfuric acid;
triethanolamine (C₆H₁₅NO₃);
Water.

According to a preferred embodiment of the invention, the composition comprises by mass, relative to the total mass of the composition:
2 parts of ricinoleic acid saponified with sulfuric acid;
1 part of triethanolamine 85% (C₆H₁₅NO₃);
2 to 10 parts of water.

This mixture of chemical compounds comprises a group of fatty acids and amino acids that are essential for plant development.

The composition according to the invention can also comprise other types of nutrients, known in the art, depending on the type of crop to be treated.

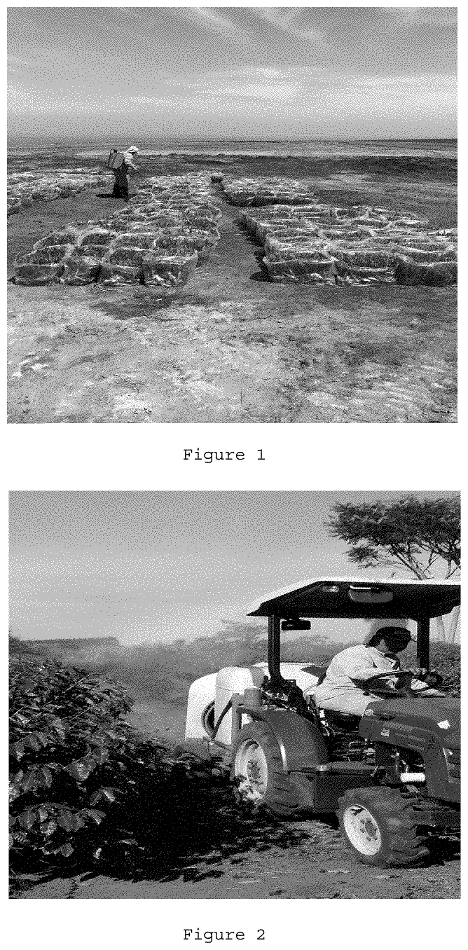

The final product, according to the invention, will be a liquid that can be applied by spraying onto the leaves of the plants, as shown in FIGS. 1 and 2.

The dilution of the final product for application in sprayed crops can vary from 1/600 to 1/1200 depending on the crop.

Tests were carried out to prove the validity of the information mentioned above, and the improvement in productivity was confirmed.

The tests were carried out with different kinds of cultures, in order to verify the efficiency of the composition of the invention in the different crops.

The following examples are merely illustrative of the scope of the invention, and should not be used for limiting purposes.

### Example 1

Production process for 200 liters of ricinoleic acid saponified with sulfuric acid.

Raw materials used:

TABLE 1

| Substance | Amount |
|---|---|
| 100% Commercial Sodium Hydroxide in | 30 kg |
| flakes | |
| Extra pale castor oil | 190 kg |
| 98% sulfuric acid | 40 kg |
| Ethanol | 15 liters |
| Ice | 35 kg |
| Tap water | |

The process comprised the following steps:
1) Dilute the Sodium Hydroxide directly in the tank in 200 liters of warm water (35° to 45° C.). Mix until dissolved.
2) Add Extra Pale Castor Oil.
3) Add ethanol.
4) Stir at high speed for 15 minutes.
5) Turn off the stirring and leave to rest for 10 minutes.
6) Stir at high speed for 25 minutes.
7) In a separate container, dilute the sulfuric acid in 35 kg of ice.
8) With the stirrer off, add the sulfuric acid+ice mixture (item 7).
9) Stir for 5 minutes.
10) Stop stirring and wait for phase separation (10 to 20 minutes).
   If phase separation does not occur within the indicated times, add sulfuric acid in batches of 5 kg, stir for 15 minutes and wait for separation.
11) Drain the lower phase (discard)
12) Add 100 liters of water.
13) Stir for 15 minutes.
14) Stop stirring and wait for phase separation (10 to 20 minutes).
   If phase separation does not occur within the indicated times, add sulfuric acid in batches of 5 kg, stir for 15 minutes and wait for separation.
15) Drain the lower phase (discard).
16) Add 100 liters of water.
17) Stir for 15 minutes.
18) Stop stirring.
19) Wait for complete phase separation (30 to 90 minutes).
20) Completely drain the lower phase (discard).
21) At the end of this process about 200 l of ricinoleic acid saponified with sulfuric acid are obtained.

### Example 2

The ricinoleic acid saponified with sulfuric acid obtained in example 1, is used to produce a fertilizer in liquid form by adding triethanolamine and water in the ratios indicated below:
2 parts of ricinoleic acid saponified with sulfuric acid;
1 part of triethanolamine 85% (C₆H₁₅NO₃);
2 parts of water.

In the following examples, the fertilizer composition according to the invention, obtained by dilution in water at the ratio of 1/600 of product and water, respectively, was used.

### Example 3

The test was carried out with coffee crop, in the region of São Sebastão do Paraiso, State of Minas Gerais, on a private property, with an increase in productivity of approximately 14% in the amount of bags of coffee produced, related to control trees.

The amounts produced, both in the control trees and in the plants where the product was applied, can be seen in Table 2. The variety of coffee in which the tests were carried out is Mundo Novo. The crop in question is the crop harvested in July 2018.

TABLE 2

| | | | Production per |
|---|---|---|---|
| | Number of | Production | 1000 plants |
| Type of plant | plants | (bags) | (bags) |
| Plant with | 6000 | 31.33 | 5.22 |
| compound | | | |
| application | | | |
| control plant | 4000 | 18.34 | 4.59 |

It is important to emphasize that each bag of coffee weights 60 kilos and that the variety and age of the coffee crops are the same, both for the control planta and the treated plants.

### Example 4

The test was carried out in the Bauru region, in the State of São Paulo, also on a private property. The crop was orange.

The harvest is from November 2018. The values obtained in the tests can be seen in Table 3. These tests showed an increase in productivity of approximately 16% in the number of orange boxes produced by the treated trees compared to the control tree.

TABLE 3

| | Number of | Production | Production per |
|---|---|---|---|
| Type of plant | plant (trees) | (boxes) | 1 plant (boxes) |
| Plant with | 988 | 1853 | 1.876 |
| compound | | | |
| application | | | |
| control plant | 3504 | 5675.9 | 1.620 |

The tests performed were carried out with oranges of the same variety and age, both for the treated and the control trees. Each orange box used in the tests had a weight of 48 kilograms.

### Example 5

The test was carried out in the region of Barra Bonita, State of Slo Paulo, on another private property, in sugarcane crop, with pre-sprouted seedlings, of CTC 9001 variety.

The tests were carried out in November 2017. It was verified in this test an increase of 13% of dry vegetative mass in the treated seedlings, as compared with the control seedlings. The values obtained in the test can be seen in Table 4.

TABLE 4

| | Number of | Quantity of | |
|---|---|---|---|
| type of | seedlings | seedlings | Production |
| seedling | (trays) | (per tray) | (grams) |
| Seedlings | 25 | 162 | 260.87 |
| with | | | |
| compound | | | |
| application | | | |
| control | 25 | 162 | 230.90 |
| seedlings | | | |

The production values were collected by sampling, two trays of each type of seedling crop were collected (treated and control). The test was performed with crops of the same age and variety. The production values were measured after the drying period in a natural drying oven.

While the invention has been described related to a preferred embodiment, it shall be understood that it is not intended to limit the invention to that particular embodiment. Therefore, it shall intend to cover all possible alternatives and equivalent modifications within the spirit and scope of the invention.

1. Process for obtaining a liquid foliar fertilizer for spraying onto the leaves of plants wherein said process comprises the following steps:
(a) in a first container dilute a strong base in water until reaching a mass concentration of about 5% to about 25% at a temperature between about 30° C. and about 50° C. and stir until complete dissolution;
(b) add castor oil in the ratio of about 70% to about 90% of the mass of item (a) above and an alcohol in the ratio of about 3% to about 12% of the mass of item (a) above, stirring strongly, leaving to rest and then stirring again
(c) in a second container dilute sulfuric acid in water up to about 40% to about 65% by weight at a temperature of ° C.;
(d) without stirring, add to the mixture of the second container the mixture of the first container, followed by stirring;
(e) stop the stirring and leave to rest; when phase separation occurs drain the lower phase and discard it;
(f) to the product obtained in step (e) add water in an amount of about 30% to about 55% of the mass of item (a) above, stir, and leave to rest; after phase separation drain the lower phase (waste) and discard it;
(g) to the product obtained in the previous step add water in an amount of about 30% to about 55% of the mass of item (a) above, stir, and leave to rest; after phase separation, discard the lower phase;
(h) add triethanolamine in a container, then water and later the product obtained in the previous step (g), in the following ratio by mass:
2 parts ricinoleic acid saponified with sulfuric acid;
1 part triethanolamine (C₆H₁₅NO₃);
2 to 10 parts water.

2. Process according to claim 1, wherein the strong base is sodium hydroxide.

3. Process according to claim 1, wherein the alcohol is ethanol.

4. Process according to claim 1, wherein said process comprises the following steps:
(a) in a container dilute 100% sodium hydroxide in flakes in water to reach concentration of 13.04% by mass at a temperature between about 35° C. and about 45° C. and stir until dissolved;
(b) add extra pale castor oil in the ratio of 82.61% of the mass of item (a) above and ethanol in the ratio of 6.52% of the mass of item (a) above, under stronger stirring for 15 min, leave to rest for 10 min and then stir again for 25 min;
(c) in another container dilute 98% sulfuric acid in water to reach a mass concentration of 52.27% at a temperature of about 0° C.;
(d) add to the mixture obtained in the previous step (c) to the mixture obtained in step (b), without stirring, and then stir for 5 min;
(e) turn off the stirring and leave to rest for 10 to 20 min; after phase separation drain and discard the lower phase;
(f) to the product obtained in step (e) add water in an amount of 43.48% of the mass of item (a) above, stir for 15 min and leave to rest for 10 to 20 min; after phase separation), drain and discard the lower phase;
(g) to the product obtained in the previous step, water is added in an amount of 43.48% of the mass of item (a) above; and stir for 15 min, then leave to rest for 30 to 90 min: after phase separation, drain and reject the lower phase;
(h) pour triethanolamine in a container, then water and later the product obtained in the previous step (g), in the following ratio by mass:
2 parts ricinoleic acid saponified with sulfuric acid;
1 part triethanolamine (C₆H₁₅NO₃) 85%;
2 to 10 parts water.

5. Liquid fertilizer composition for foliar application, wherein said composition comprises:
ricinoleic acid saponified with sulfuric acid;
triethanolamine (C₆H₁₅NO₃);
Water.

6. Liquid fertilizer composition according to claim 5, wherein said composition comprises:
2 parts ricinoleic acid saponified with sulfuric acid;
1 part triethanolamine 85% (C₆H₁₅NO₃);
2 to 10 parts water.

7. Liquid fertilizer composition according to claim 5, wherein said composition further comprises other types of nutrients suitable for each type of crop.

8. Liquid fertilizer composition according to claim 5, wherein said composition is diluted in water in a ratio of 1/600 to 1/1200 before spray application on crops.
